(19) United States
(12) Patent Application Publication
(10) Pub. No.: US 2026/0204101 A1
(43) Pub. Date: Jul. 16, 2026

(54) SYSTEM AND METHOD FOR ANALYZING AND CORRECTING GAIT BASED OFF CAPTURED IMAGES

(71) Applicants: Eric M. Greber, Thibodaux, LA (US); Kristen E. Martin, Elkhart, IN (US); Sebastien Parratte, Abu Dhabi (AE)

(72) Inventors: Eric M. Greber, Thibodaux, LA (US); Kristen E. Martin, Elkhart, IN (US); Sebastien Parratte, Abu Dhabi (AE)

(21) Appl. No.: 19/179,334

(22) Filed: Apr. 15, 2025

Related U.S. Application Data
(60) Provisional application No. 63/633,929, filed on Apr. 15, 2024.

(51) Int. Cl. G06V 40/20; A61B 5/11; G16H 20/30

(52) U.S. Cl. CPC G06V 40/25; A61B 5/112; G16H 20/30

(57) ABSTRACT

A system for addressing gait abnormalities includes an image capture system having at least one image capture device configured to capture one or more images of a patient and a processing system having at least one processor operatively coupled to the image capture system. The processing system is configured to: analyze the one or more images of the patient; construct a digital wireframe model approximating a skeleton of the patent based on the one or more images; determine one or more anatomical angles in the digital wireframe model; determine one or more gait abnormalities exists based on the determined one or more anatomical angles deviating from a defined value by a threshold value; and provide a suggestion to correct the one or more determined gait abnormalities based on the one or more anatomical angles that deviates from the defined value by the threshold value.

### CROSS-REFERENCE TO RELATED APPLICATIONS

This is a non-provisional application based upon U.S. Provisional Patent Application Ser. No. 63/633,929 entitled “SYSTEM AND METHOD FOR ANALYZING AND CORRECTING GAIT BASED OFF CAPTURED IMAGES,” filed on Apr. 15, 2024, which is incorporated in its entirety herein by reference.

### BACKGROUND OF THE INVENTION

### 1. Field of the Invention

The present invention relates to systems and methods for analyzing and correcting gait.

### 2. Description of the Related Art

Osteo-arthritis (OA) and various orthopaedic conditions impact the gait pattern. After surgery, patients experience difficulties returning to a normal gait pattern. Today's therapeutic practices are collecting biometrics but not the quality of the gait pattern. Actual quantitative and qualitative gait analysis requires the complex set-up of a gait lab limiting the clinical use of such tools. Today, during the daily clinical orthopedics practice, quantitative and qualitative gait analysis is still under-utilized.

### SUMMARY OF THE INVENTION

The present invention provides a system for analyzing and correcting gait that includes one or more processors configured to analyze one or more captured images and, based on the images, identify one or more gait abnormalities, and provide suggestions on how to reduce and/or eliminate the one or more gait abnormalities.

In some embodiments provided according to the present invention, a system for addressing gait abnormalities includes an image capture system having at least one image capture device configured to capture one or more images of a patient and a processing system having at least one processor operatively coupled to the image capture system. The processing system is configured to: analyze the one or more images of the patient; construct a digital wireframe model approximating a skeleton of the patent based on the one or more images; determine one or more anatomical angles in the digital wireframe model; determine one or more gait abnormalities exists based on the determined one or more anatomical angles deviating from a defined value by a threshold value; and provide a suggestion to correct the one or more determined gait abnormalities based on the one or more anatomical angles that deviates from the defined value by the threshold value.

In some exemplary embodiments provided according to the present invention, a method of addressing gait abnormalities includes: capturing one or more images of a patient using an image capture system including at least one image capture device; analyzing the one or more images of the patient; constructing a digital wireframe model approximating a skeleton of the patent based on the one or more images; determining one or more anatomical angles in the digital wireframe model; determining one or more gait abnormalities exists based on the determined one or more anatomical angles deviating from a defined value by a threshold value; and providing a suggestion to correct the one or more determined gait abnormalities based on the one or more anatomical angles that deviates from the defined value by the threshold value.

One possible advantage that may be realized by embodiments provided according to the present invention is that patient gait abnormalities can be readily identified and corrected in a non-invasive manner.

Another possible advantage that may be realized by embodiments provided according to the present invention is that one or more healthcare providers can use the system to monitor patient progress or lack thereof and adjust the treatment plan accordingly.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a schematic view of an exemplary embodiment of a system for addressing gait abnormalities provided according to the present invention;

FIG. 2 is a captured image of a patient, taken from in front of the patient, displaying multiple anatomical angles and a gait abnormality determined according to the present invention;

FIG. 3 is a captured image of the patient, taken from a side of the patient, displaying multiple anatomical angles determined according to the present invention with no associated gait abnormality;

FIG. 4 is an exemplary graphical user interface that may be presented on a display of the system of FIG. 1 to analyze various aspect of the patient's gait according to the present invention;

FIG. 5 is another captured image of the patient, taken from a side of the patient, displaying multiple anatomical angles determined according to the present invention with no associated gait abnormality;

FIG. 6 is another captured image of the patient, taken from in front of the patient, displaying multiple anatomical angles and a gait abnormality determined according to the present invention;

FIG. 7 is another captured image of the patient, taken from a side of the patient, displaying a gait length of the patient determined according to the present invention;

FIG. 8A is another captured image of the patient, taken from a side of the patient, displaying the patient having a heel strike determined according to the present invention;

FIG. 8B is another captured image of the patient, taken from a side of the patient, displaying the patient having a mid foot strike determined according to the present invention;

FIG. 8C is a schematic illustration of a wireframe model of a patient displaying a fore foot strike;

FIG. 8D illustrates different foot strike patterns exhibited in FIGS. 8A-8C;

FIG. 9 is another captured image of the patient, taken from a side of the patient, displaying a gait length of the patient determined according to the present invention;

FIG. 10A is an exemplary graphical user interface that may be presented on the display of the system of FIG. 1 indicating to the patient that the captured images of the patient's gait did not indicate a gait abnormality;

FIG. 10B is an exemplary analysis screen that may be presented on the display of the system of FIG. 1 indicating to the patient that the captured images of the patient's gait indicate a gait abnormality and providing a suggestion on how to correct the gait abnormality;

FIG. 11A is an exemplary embodiment of an analysis screen that may be presented on the display of the system of FIG. 1 that shows the determined gait abnormality and a suggestion to correct the gait abnormality;

FIG. 11B is an exemplary embodiment of an analysis summary screen that may be presented on the display of the system of FIG. 1 providing a summary of the patient's gait;

FIG. 12 is an exemplary embodiment of a post-operative gait analysis screen that may be presented on the display of the system of FIG. 1 that shows the determined anatomical angles and other physiological markers determined from the recorded images captured by the imaging device of the system; and

FIG. 13 is an illustration of another anatomical angle, which is known as knee flexion, that can be determined and used to identify gait abnormalities according to the present invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

The present invention provides a system and method that can identify gait abnormalities in a patient and provide suggestions on how to correct the one or more gait abnormalities, i.e., address gait abnormalities. The system includes one or more processors that are configured to analyze one or more images of a patient, construct a digital wireframe model approximating the patient's skeleton based on the image(s), determine one or more anatomical angles in the digital wireframe model approximating the patient's skeleton, determine one or more gait abnormalities exists based on the determined one or more anatomical angles deviating from a defined value by a threshold value, and provide a suggestion to correct the one or more determined gait abnormalities based on the one or more anatomical angles that deviate from the defined value by the threshold value. Similarly, the present invention provides a method that includes analyzing one or more images of a patient, constructing a digital wireframe model approximating the patient's skeleton based on the image(s), determining one or more anatomical angles in the digital wireframe model approximating the patient's skeleton, determining one or more gait abnormalities exists based on the determined one or more anatomical angles deviating from a defined value by a threshold value, and providing a suggestion to correct the one or more determined gait abnormalities based on the one or more anatomical angles that deviate from the defined value by the threshold value. The method may be performed by the system.

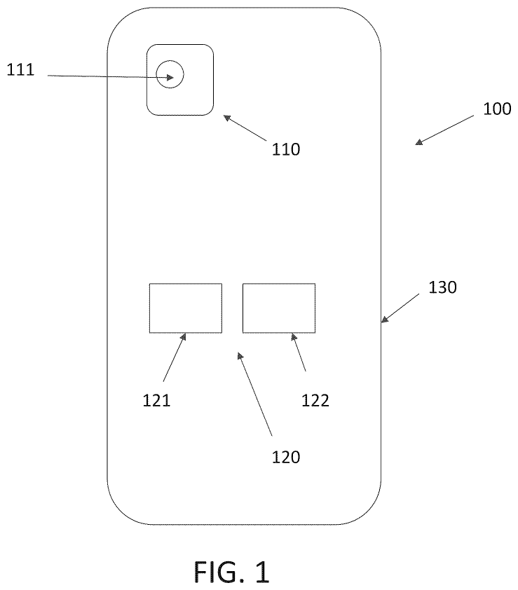

Referring now to the drawings, and more particularly to FIG. 1, there is shown an exemplary embodiment of a system 100 provided according to the present invention which generally includes an image capture system 110 including at least one image capture device 111 and a processing system 120 including at least one processor 121 that is operatively coupled to the image capture system 110. In some embodiments, as illustrated, the system 100 includes a common housing 130 that houses both the image capture system 110 and the processing system 120. The system may, for example, comprise a smartphone, a digital tablet, a laptop computer, or another similar device that incorporates both an image capture system and a processing system. However, it should be appreciated that the image capture system 110 may be housed separately from the processing system 120, with the image capture system 110 and the processing system 120 operatively coupled to one another by a data connection, e.g., a wired or wireless communication interface.

The image capture system 110 is configured to capture one or more images of a patient using the image capture device(s) 111. In some embodiments, the image capture device(s) 1111 consists of a single image capture device, such as a camera, that is configured to capture photographic images of the patient. Such cameras are well-known in the art and may be configured to take still images, i.e., single images, and/or moving images, i.e., a series of images, according to the present invention. While the system 100 illustrated in FIG. 1 illustrates an image capture system 110 with a single image capture device 111, it should be appreciated that the image capture system 110 may include multiple image capture devices, e.g., multiple cameras arranged at different locations, in order to capture images from different perspectives. It should be further appreciated that the image capture device(s) 111 is described as being a photographic image capture device but may be a different type of image capture device, e.g., a thermal imaging device. It should thus be appreciated that different types of image capture devices may be provided according to the present invention.

The images captured by the image capture device(s) 111 may be processed by a graphical processor 122, which may also be referred to as a graphical processing unit (GPU), that is coupled to (or part of) the image capture device(s) 111 and/or part of the processing system 120. In some embodiments, the GPU 122 is a distinct processor that is part of the processing system 120. The GPU 122 may be configured to analyze the captured image(s) and perform other graphics-related analysis, as described further herein. Many types of GPUs are known in the art and may be provided according to the present invention. The processing system 120 may also include a central processor, which is illustrated as processor 121 and may also be referred to as a central processing unit (CPU), that is configured to perform operations separately from the GPU 122. In some embodiments, the CPU 121 is separate from the GPU 122 but it should be appreciated that, in some embodiments, the CPU 121 may perform the graphical operations described herein as being performed by the GPU 122. It should thus be appreciated that the processing system 120, generally, has one or more processors/processing units 121, 122 that are configured to perform the operations described herein. It should be further appreciated that a processing system having multiple processors may include processors that are not disposed in a common housing and are merely operatively coupled to one another via a communication interface.

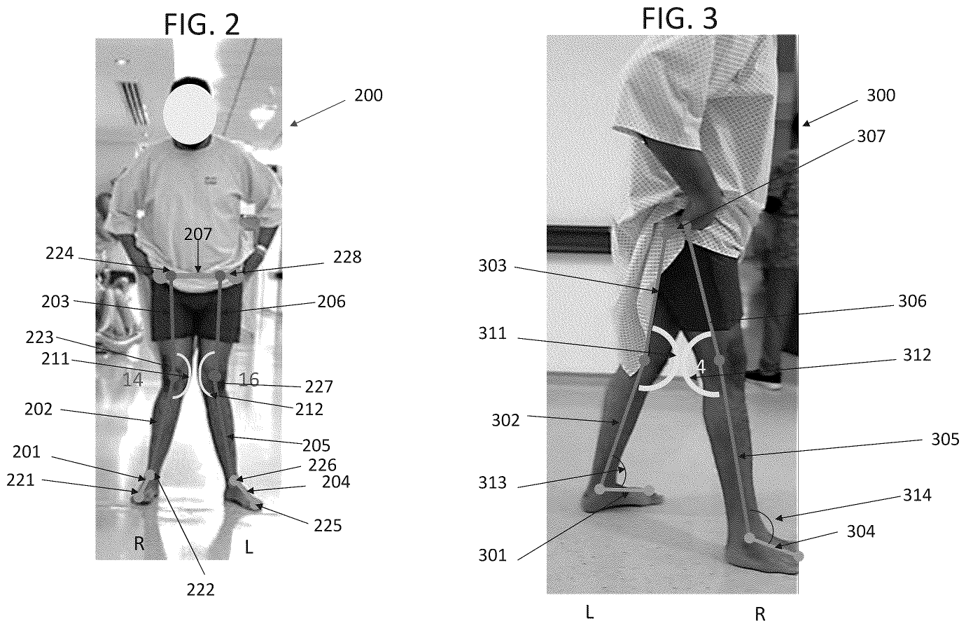

As previously described, the image capture system 110 is configured to capture one or more images of a patient for analysis. The captured image(s) may, for example, be multiple images captured from a video recording of a patient as the patient is walking. As illustrated in FIGS. 2 and 3, the image(s) 200, 300 may be captured from a first perspective (FIG. 2), which may be a front-on (coronal plane) perspective of the patient walking, and also may be captured from a second perspective (FIG. 3), which may be a side (sagittal plane) perspective of the patient walking. After the image(s) 200, 300 is captured, the processing system 120 is configured to analyze the captured image(s) 200, 300 and construct a digital wireframe model approximating the patient's skeleton based on the image(s) 200, 300, as shown in FIGS. 2 and 3. It should be appreciated that the digital wireframe model approximating the patient's skeleton does not need to approximate the entire skeleton, i.e., the digital wireframe model may approximate a portion of the skeleton including one or more bones and/or joints. It should be further appreciated that the digital wireframe model “approximates” the patient's skeleton because the wireframe model may be constructed as, for example, one or more connected line segments 201, 202, 203, 204, 205, 206, 207, 301, 302, 303, 304, 305, 306, 307 which may not show curvatures and other aspects of the natural anatomical shape of the one or more approximated bones and/or joints of the skeleton. The GPU 122 may be configured to analyze the captured image(s) 200, 300 and also construct the digital wireframe model approximating the patient's skeleton, as both of these operations are graphics-intensive processes; however, it should be appreciated that the CPU 121 may also be configured to perform these operations alone or jointly with the GPU 122. Once the digital wireframe model approximating the patient's skeleton is constructed, the processing system 120 may determine one or more anatomical angles 211, 212, 311, 312, 313, 314 in the digital wireframe model approximating the patient's skeleton, as also illustrated in FIGS. 2 and 3. For example, the processing system 120 may determine one or more anatomical angles 211, 212, 311, 312, 313, 314 in the digital wireframe model approximating the patient's skeleton at regions that are recognized as joints, such as the hips, knees, and ankles.

The processing system 120 may be configured in a variety of ways to construct the digital wireframe model approximating the patient's skeleton and determine one or more anatomical angles 211, 212, 311, 312, 313, 314 in the digital wireframe model approximating the patient's skeleton. The processing system 120 may be provided with instructions and/or algorithms, which may be provided in the form of software, that analyzes the images 200, 300 to construct the digital wireframe model approximating the patient's skeleton and determine the anatomical angle(s) 211, 212, 311, 312, 313, 314. For example, the processing system 120 may be provided with an algorithm that allows the processing system 120 to detect objects in images. Exemplary algorithms include, but are not limited to, algorithms for pose estimation based on the You Only Look Once model, which is a deep learning model trained to detect objects (in this case, human anatomical keypoints). The processing system 120 can then use the pose estimation algorithm and mathematical operations to calculate anatomical angles and distances between various body joints. In this respect, the processing system 120 may utilize artificial intelligence to recognize human anatomical features in images and create the digital wireframe model approximating the patient's skeleton as well as determine the anatomical angle(s).

Once the anatomical angle(s) 211, 212, 311, 312, 313, 314 is determined, the processing system 120 may determine one or more gait abnormalities exists based on the determined anatomical angle(s) 211, 212, 311, 312, 313, 314 deviating from a defined value by a threshold value. As is known, anatomical angles (and other physiological markers) may vary between different people so an abnormality is typically associated with a deviation from a “normal range” that is defined based on measurements taken from healthy individuals, i.e., those without associated disease processes. In other words, the processing system 120 is configured to determine that the determined anatomical angle(s) 211, 212, 311, 312, 313, 314 lies outside of the normal range to determine that one or more gait abnormalities exists. The defined value and the threshold value may be defined based on a variety of sources, e.g., medical literature, previously measured values, etc. In some embodiments, the defined value and/or the threshold value are stored in a memory of the system 100 that is operatively coupled to the processing system 120. The defined value and/or the threshold value may be loaded into the memory by the processing system 120, e.g., by user input to the CPU 121, or may be pre-loaded into the memory.

The processing system 120 may also be configured to classify a degree of gait abnormality based on a difference between the determined anatomical angle(s) 211, 212, 311, 312, 313, 314 and the defined value. For example, the processing system 120 may be configured to classify a gait abnormality as “absent” or “neutral” if the determined anatomical angle(s) 211, 212, 311, 312, 313, 314 does not differ from the defined value by the threshold value; classify a gait abnormality as “mild” if the determined anatomical angle(s) 211, 212, 311, 312, 313, 314 differs from the defined value by the threshold value but not by a second threshold value that is greater than the threshold value; and classify a gait abnormality as “severe” if the determined anatomical angle(s) 211, 212, 311, 312, 313, 314 differs from the defined value by the second threshold value. In this respect, the processing system 120 may also be utilized to classify the degree of gait abnormality in order to provide a user with information as to how much the analyzed gait deviates from a healthy gait.

Once the processing system 120 has determined one or more gait abnormalities exists, the processing system 120 is configured to provide a suggestion to correct the one or more determined gait abnormalities based on the anatomical angle(s) that deviates from the defined value by the threshold value. It should be appreciated that, as used herein, the term “correct” does not mean that the one or more gait abnormalities must actually be eliminated; it is sufficient that the provided suggestion provides information to the patient on how to improve their gait. It should be further appreciated that whether the patient's gait ultimately improves depends on a variety of factors, including patient compliance and other behavior, the quality of data input, medical team instructions, etc. It should thus be appreciated that the system 100 provided according to the present invention is a tool that a patient can utilize to help correct one or more gait abnormalities but a successful outcome depends on factors out of the control of the system 100.

The provided suggestion may be provided in a variety of forms. The provided suggestion may be, for example, a visual signal output by the processing system 120 to a visual display incorporating pictures and/or text to assist the patient in doing the one or more activities to correct the one or more gait abnormalities. In some embodiments, the provided suggestion may be a visual signal that causes a text representation of a particular movement to take, such as “increase gait length,” as well as a video showing how much longer the gait length should be. In some embodiments, the provided suggestion incorporates a uniform resource locator (URL) that directs the patient to a web site with information about how to correct the one or more gait abnormalities. In some embodiments, the provided suggestion may be an audio signal corresponding to an audio component, which may be output to an audio device such as a speaker of the system 100 to play a sound. For example, the audio component may be a recorded sound or instruction that presents the suggestion to the patient by indicating to the patient what action to take to help correct the one or more gait abnormalities, e.g., a pre-recorded voice saying “rotate your right foot slightly counter-clockwise.” It should thus be appreciated the suggestion may be provided in a variety of ways according to the present invention.

The provided suggestion may be derived from a lookup table or other source that includes a plurality of defined suggestions each associated with a particular anatomical angle. The defined suggestions may also each be associated with a degree of gait abnormality. For example, the processing system 120 may provide a first suggestion if the processing system 120 determines that a gait abnormality exists based on an anatomical angle at the hip exceeding the defined value by the threshold value (mild abnormality) but may alternatively provide a second suggestion that differs from the first suggestion if the processing system 120 determines that the anatomical angle at the hip exceeds the defined value by the second threshold value (severe abnormality). The processing system 120 may also be configured to analyze multiple anatomical angles in order to identify the underlying pathology of the gait abnormality and provide a suggestion that addresses the pathology to correct the gait abnormality.

In some embodiments, the processing system 120 is configured to utilize a diagnostic algorithm that takes into account the anatomical angle(s), and optionally additional physiological data as well, to provide a suggestion to correct the one or more determined gait abnormalities. The processing system 120 may also be configured to utilize machine learning to improve the diagnostic algorithm based on how effective the suggestion is to correct the one or more gait abnormalities.

Based on the foregoing, it should be appreciated that the system 100 provided according to the present invention may be used both pre-operatively and post-operatively to help correct a patient's gait abnormalities. For example, the processing system 120 may be used pre-operatively to provide a healthcare provider with information about the one or more abnormalities associated with the patient's gait and suggest possible surgical interventions that could correct the abnormalities. Similarly, the system 100 can be used post-operatively (or without any intervening surgical intervention) to help a patient undergoing physical therapy identify abnormalities that may be present in their gait and corrective measures that the patient (or healthcare provider) can take to correct the abnormalities. For example, the processing system 120 may be used to identify a particular gait abnormality and suggest gait changes, exercises, stretches, or other techniques that the patient can perform in order to correct the gait abnormality. It should thus be appreciated the system provided according to the present invention can be utilized in a variety of ways to correct gait abnormalities.

### Exemplary Functionality

An example setup of a system 100 provided according to the present invention is further described herein. The system 100 may be provided, for example, in the form of a smart device such as a smartphone with a memory storing an application, commonly known as an “app,” as software that implements the functionality. In other words, a system 100 provided according to the present invention may incorporate both hardware and software to operate. The system 100 may thus be formed when hardware is loaded with instructions or code in the form of software that is tangibly stored in the memory of the system or otherwise provided to the hardware. It should be appreciated that the following description is of examples only and other embodiments may be provided according to the present invention.

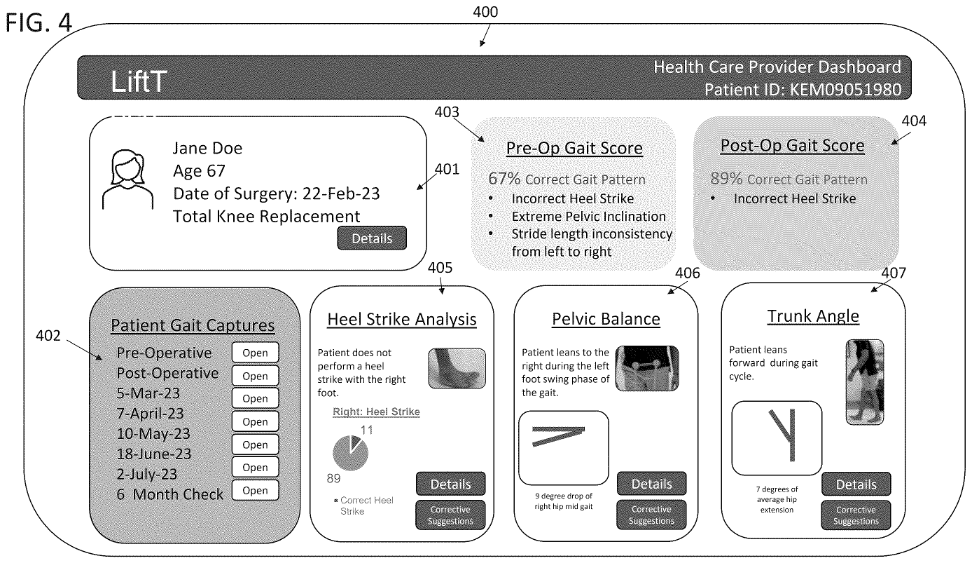

Referring to FIG. 4, an exemplary first graphical user interface (GUI) 400 of an application provided according to the present invention is illustrated. The first GUI 400 may be tailored towards use by a healthcare provider and includes a plurality of boxes including a patient information box 401, a gait capture box 402, a pre-operative gait score box 403, a post-operative gait score box 404, and a plurality of physiological markers boxes 405, 406, 407. A healthcare provider user is any user who will assess and/or treat the patient with a gait abnormality, disease of one or more joints, or other skeletomuscular injury or recovery. The healthcare provider may assess the patient prior to and/or after treatment, which may include physical therapy and/or surgery, so that they can see the angular data of the joints as well as the gait pattern. The healthcare provider user will also have post operative full access to the data collected in their office and each time the patient uploads a new video of their gait. Each time a new gait video (or multiple images) is captured, the healthcare provider user receives a notification and is able to view the video/image(s). The first GUI 400 may be provided in the application loaded into the memory coupled to the processing system 120 of the system 100 or, alternatively, may be provided in software that is loaded onto a second device that is remote from the device used to capture the image(s).

To start, the healthcare provider (or patient) may access the application of the system and initiate a new gait video capture using the imaging device(s) 111 from an icon in the gait capture box 402. Once a new gait video capture has been initiated, the application will access the imaging device(s) 111, e.g., a camera of the device, so the imaging device(s) 111 begins to capture images. The healthcare provider (or a pre-recorded sound output by the device) can ask the patient to stand a specific distance from the imaging device(s) 111. In some embodiments, the system 100 is configured to recognize that the patient is an improper distance and/or orientation relative to the imaging device(s) 111 and output an instruction, such as an audio instruction, to instruct the patient to get in the proper orientation and location for image capturing. The patient is instructed to walk toward the imaging device(s) 111 while the imaging device(s) 111 is recording. At the end of the specified walk, the healthcare provider (or the system) will stop the recording. The system 100 may, for example, be configured to recognize that the patient is a specified stopping distance from the imaging device(s) 111 and then output instructions to the patient to stop walking. The patient can then be asked to walk a specified distance across a different perspective while a second recording is captured by the imaging device(s) 111, as previously described with respect to FIGS. 2-3. After the recording is complete, the processing system 120 can analyze the captured image(s) and prepare information for review. In some embodiments, review is available without any significant delay and can be viewed momentarily after recording, e.g., within 30-60 seconds of recording.

The processing system 120 may be configured to calculate and analyze a variety of angles and other data points from the recorded images. The following data points are exemplary and represent just some of the possible data that can be analyzed and calculated from the recorded images to help correct the patient's gait.

### Hip Knee Ankle Angle

The processing system 120 may be configured to calculate a hip knee ankle angle as the one or more anatomical angles described previously. FIG. 2 illustrates an image 200 that may be utilized by the system provided according to the present invention to calculate the hip knee ankle angle 211, 212. In some embodiments, the relevant metrics for calculating the hip knee ankle angle 211, 212 are captured in one or more images taken in the coronal plane, i.e., with the patient walking towards the imaging device(s) 111 as illustrated. An angle 211, 212 is measured on the inside of each knee between the femoral axis (illustrated as line segments 203 and 206) and the tibial axis (illustrated as line segments 202 and 205), as illustrated in FIG. 2, to define the hip knee ankle angle 211, 212. If the calculated hip knee ankle angle 211, 212 is less than 180 degrees, then the leg is in varus. If the angle 211, 212 is greater than 180 degrees, the leg is in valgus. The defined value for the hip knee ankle angle 211, 212 may thus be 180 degrees, with a determined hip knee ankle angle 211, 212 that deviates from 180 degrees by a threshold value indicating that one or more gait abnormalities exists.

The system 100 may be configured so a value shown in the application is the angle between the femur 203, 206 and tibia 202, 205 on the inside of the knee if varus is detected. The value can be displayed for the user in terms of varus degrees of deformity, i.e., the negative deviation from the defined value. Alternatively, if valgus is detected, the value shown in the app can be the angle between the femur 203, 206 and tibia 202, 205 on the outside of the knee. The value can be displayed for the user in terms of valgus degrees of deformity, i.e., the positive deviation from the defined value. It should be appreciated that the hip knee ankle angle 211, 212 changes throughout the gait, therefore, it may only be displayed on the final analysis at the time of the heel strike.

The following Table 1 illustrates varying degrees to which the hip knee ankle angle may deviate from the defined value.

TABLE 1

| | Neutral | Mild | Severe |
|---|---|---|---|
| Varus Angle | 0-3 degrees | >3-7 degrees | >7 degrees |
| Valgus Angle | 0-3 degrees | >3-7 degrees | >7 degrees |
| Indicator | Green Line | Amber Line | Red Line |

As can be seen from Table 1, the system 100 can be configured to determine that one or more gait abnormalities exist if the determined hip knee ankle angle 211, 212 deviates from the defined value, e.g., 180 degrees, by more than 3 degrees. In some embodiments, the system 100 is configured to display a visual indicator of the one or more gait abnormalities being present by overlaying dots 221, 222, 223, 224, 225, 226, 227, 228 over joints in one or more captured images 200 (See FIG. 2) and connecting the dots with line segments 201, 202, 203, 204, 205, 206 representing the rays of the angles, e.g., the hip knee ankle angle 211, 212. The connected line segments 201, 202, 203, 204, 205, 206 may be different colors to indicate the degree of the deviation, as illustrated in Table 1. FIG. 2 illustrates that the determined anatomical angle deviations are 14 degrees at the right leg and 16 degrees at the left leg, indicating a severe degree of gait abnormality and presenting the respective line segments 202, 203, 205, 206 in red as a visual representation.

### Sagittal Knee Angle at Heel Strike (Flexion Angle)

The processing system 120 may be configured to determine the sagittal knee angle at heel strike, also known as the “flexion angle,” as the one or more anatomical angles, which is illustrated as angles 311 and 312 in FIG. 3. The flexion angle 311, 312 is used to measure if the knee is straight or bent when the heel strikes in the gait. A bent knee indicates that the knee is not in full extension and there is a flexion contracture, which is not ideal.

To determine the flexion angle 311, 312, the patient gait may be recorded in the sagittal plane, i.e., as the patient walks across the screen, as shown in FIG. 3. The flexion angle 311, 312 is the angle between the 180 degrees horizontal axis of the femur and the tibia angle. A measured flexion angle of 180 degrees indicates a straight leg with no contracture, while a positive deviation indicates a flexion contracture where a patient is unable to fully extend the knee and a negative deviation indicates hyperextension where the patient is overextending the knee. When in flexion, the angle will be measured at the back of the leg. If the knee goes into hyperextension, the angle will be measured from the front of the leg. The flexion angle changes throughout the gait; therefore, it will only be displayed for final analysis with measurement taken at the time of the heel strike.

For the flexion angle, the defined value may be 180 degrees, which corresponds to a straight leg with no contracture. As shown in the following Table 2, the threshold value for deviation from the defined value may be more than 5 degrees (positive or negative) to indicate one or more gait abnormalities of a mild severity. It should be appreciated that a “negative” degree angle indicates a direction opposite to a “positive” degree angle. More than a 15 degree deviation from the defined value may be used to indicate a severe deviation. As illustrated in FIG. 3, the measured deviation is 4 degrees, which is a neutral deviation.

TABLE 2

| | Neutral | | Mild | | Severe | |
|---|---|---|---|---|---|---|
| Knee Flexion contracture | 0-5 | Degrees | >5 to 15 | degrees | >15 | degrees |
| Knee Hyper Extension | 0 to −5 | Degrees | >−5 to −15 | degrees | >−15 | degrees |

| | | | |
|---|---|---|---|
| Indicator | Green | Amber | Red |

### Ankle Sagittal Angle

The processing system 120 may be configured to determine an ankle sagittal angle as the one or more anatomical angles.

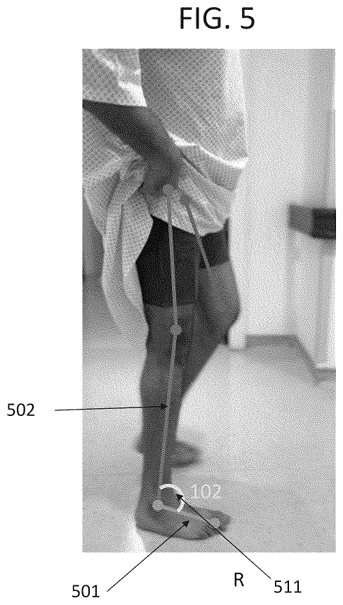

To determine the ankle sagittal angle 511, the metrics may be taken in the sagittal plane, as illustrated in FIG. 5. The ankle sagittal angle is the angle between the axis of the foot, illustrated as line segment 501, and the axis of the tibia when the foot is planted on the ground mid gait, illustrated as line segment 502. The ankle sagittal angle 511 is measured only on the foot that is planted on the ground while the other foot is mid gait and indicates whether or not the patient's gait is compromised by an inflexible ankle.

The defined value for the ankle sagittal angle may be 100 degrees. An ankle sagittal angle of at least 100 degrees indicates a healthy gait while an ankle sagittal angle of less than 100 degrees indicates a gait abnormality. Thus, the threshold value for the ankle sagittal angle may be any negative deviation from the defined value of 100 degrees. As illustrated in FIG. 5, the measured ankle sagittal ankle 511 is 102 degrees, which does not indicate a gait abnormality.

### Pelvic Inclination

The processing system 120 may be configured to determine a pelvic inclination as the one or more anatomical angles. The pelvic inclination can generally be assessed to identify dysfunction with a patient's abductor muscles. An abnormal pelvic inclination can clinically present as a “waddle” when the patient walks, which correlates with some adaptive movement by the patient to clear the foot from hitting the ground during the swing phase.

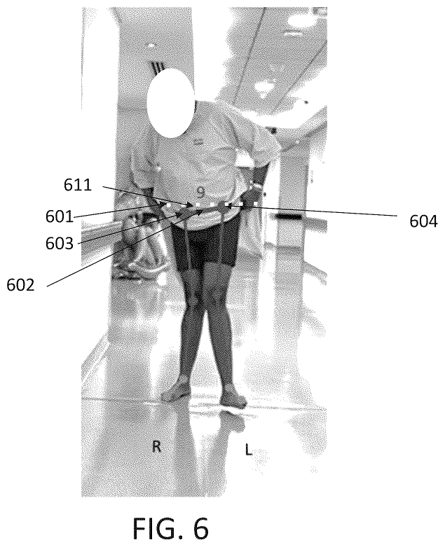

To determine the pelvic inclination, the metrics may be taken in the coronal plane, as illustrated in FIG. 6. The pelvic inclination is the angle 611 formed by a plane 601 that is parallel to the ground and an axis 602 that extends from anterior superior iliac spine (ASIS) 603 to ASIS 604 during the gait. The pelvic inclination 611 can be classified according to the particular leg stance, i.e., right leg stance and left leg stance, as indicated in the following Table 3. In other words, the pelvic inclination deviation may have a directional component as well as a numerical component. The defined value may be 0 degrees and the threshold value can be 3 degrees of deviation from the defined value, with 3-7 degrees of deviation indicating mild deviation and more than 7 degrees of deviation indicating severe deviation. As illustrated in FIG. 6, the measured pelvic inclination is 9 degrees, indicating severe deviation.

TABLE 3

| | Right Leg Stance | Left Leg Stance |
|---|---|---|
| Range | Angle of pelvis (drop of left hip) | Angle of pelvis (drop of right hip) |
| Indicator | 0-3-Neutral, >3-7-Mild, >7 Poor | 0-3-Neutral, >3-7-Mild, >7 Poor |
| | Changes 0-3 degrees-Indicate green | Changes 0-3 degrees-Indicate green |
| | Change between >3-7 degrees-Indicate Amber | Change between >3-7 degrees-Indicate Amber |
| | Change greater than 7-Indicate Red | Change greater than 7-Indicate Red |

### Other Calculations

The processing system provided according to the present invention may also calculate other potentially relevant physiological markers. Exemplary other potentially relevant physiological markers include, but are not limited to, gait length, strike type, and hip extension angle.

### Gait Length

To calculate the gait length, the metrics may be taken in the sagittal plane, as illustrated in FIG. 7. The gait length is an average distance 711 measured from a back of the heel of the left foot 701 to the back of the heel of the right foot 702. The processing system 120 can be configured to determine if there is a difference in gait length for steps that are left foot forward and right foot forward, i.e., asymmetry, which can indicate a gait abnormality. The defined value can be 0% asymmetry, indicating that the steps are the same length and a healthy gait, with a threshold value of 20%, as indicated in Table 4 below. 20% of asymmetry can indicate a mild deviation while 50% of asymmetry can indicate a severe deviation.

TABLE 4

| | MEASUREMENT |
|---|---|
| Left Step Length | Feet and inches/meters and centimeters |
| Right Step Length | Feet and inches/meters and centimeters |
| | If left and right steps <20% asymmetric- |
| | Green Indicator |
| | If left and right steps are between >20-50% |
| | asymmetric-Amber Indicator |
| | If left and right steps are >50% asymmetric - |
| | Red Indicator |

### Strike Type

The processing system can be configured to determine a patient's strike type, i.e., heel strike, mid foot strike, or fore foot strike, during a patient's gait.

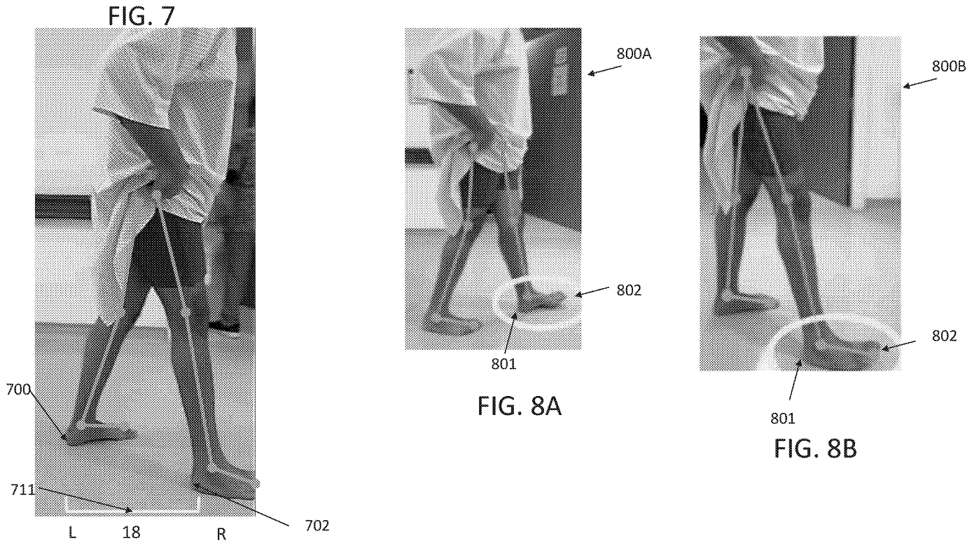

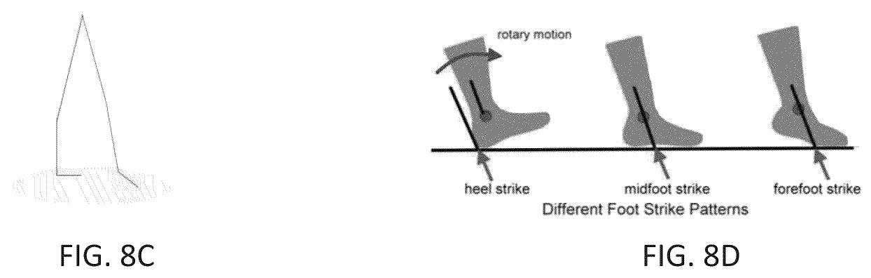

To determine the strike type, the metrics are taken in the sagittal plane, as illustrated in FIGS. 8A-8D. The processing system 120 may be configured to recognize the heel 801 and the toe 802 of the patient in the captured image(s) 800A, 800B as well as the ground and determine what part of the foot hits the ground during a step. The patient walking so their heel/hind-foot strikes first is classified as a heel strike type and is indicative of a healthy gait, i.e., the heel strikes first as the foot advanced is planted on the floor. A patient walking so their mid-foot or toe strikes the ground first, as illustrated in FIGS. 8B and 8C respectively, is indicative of one or more gait abnormalities, which the processing system 120 can recognize and provide suggestions on how the patient can alter their gait so their heel strikes first. FIG. 8D shows a comparison of the different foot strike patterns side-by-side.

### Hip Extension

The processing system 120 can be configured to determine a patient's hip extension during their gait. The hip extension indicates whether the patient is walking upright or leaning forward in their gait; this can be an indication of the spinopelvic relationship as well as hip pathology.

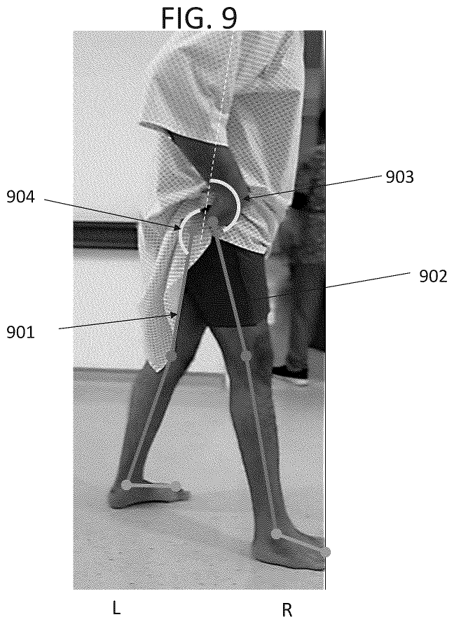

To determine the hip extension, the metrics are taken in the sagittal plane, as illustrated in FIG. 9. The hip extension 903 of the right hip is the difference between the femoral axis 901, 902 of each leg when the left leg is in heel strike and the angle of the trunk to the right femur. Similarly, the hip extension 904 of the left hip is the difference the femoral axis 901, 902 of each leg when the right leg is in heel strike and the angle of the trunk to the left femur. If the patient is not walking with a heel strike type, the determination of the hip extension can be taken at the maximum difference in the gait cycle. The measured hip extension angle may be measured and displayed to assist the user and healthcare providers in identifying one or more gait abnormalities and taking corrective measures.

### Static Knee Range of Motion

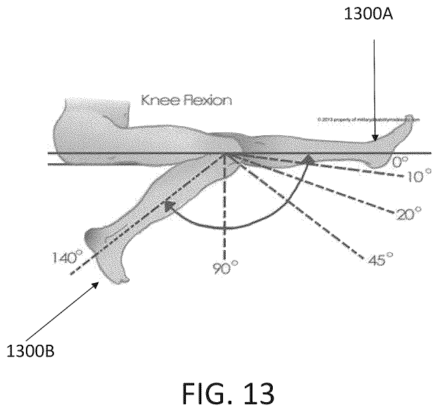

Another anatomical angle that can be determined by the processing system 120 is the static knee range of motion, which is illustrated in FIG. 13. The static knee range of motion refers to the degree of movement available at the knee joint when the leg is stationary, i.e., without active movement from the individual. The static knee range of motion can assess how far the knee can bend (flexion) or straighten (extension), and is often measured in degrees. To determine the static knee range of motion, the patient lies down on a flat surface, such as a table or bed, in a relaxed position. The image capture device(s) 111 can then capture movement of the knee as it moves to its full extension (straightening indicated by the foot position 1300A) and flexion (bending indicated by the foot position 1300B). The processing system 120 can determine the static knee angle 1301 for both extension and flexion by comparing the tibial axis to the horizon, as illustrated. A normal value for knee extension is 0°, i.e., fully straight, as illustrated for foot position 1300A, and a normal value for knee flexion is an angle range of 135-150°, with the foot position 1300B demonstrating a knee flexion angle 1301 of approximately 140°. Any deviation from these values for extension and/or flexion may lead to the presence of a gait abnormality, particularly a deviation from the values for the extension.

After the data is obtained and relevant measurements calculated, the processing system 120 can populate the various boxes of the GUI 400 illustrated in FIG. 4 with the relevant measurements and determinations.

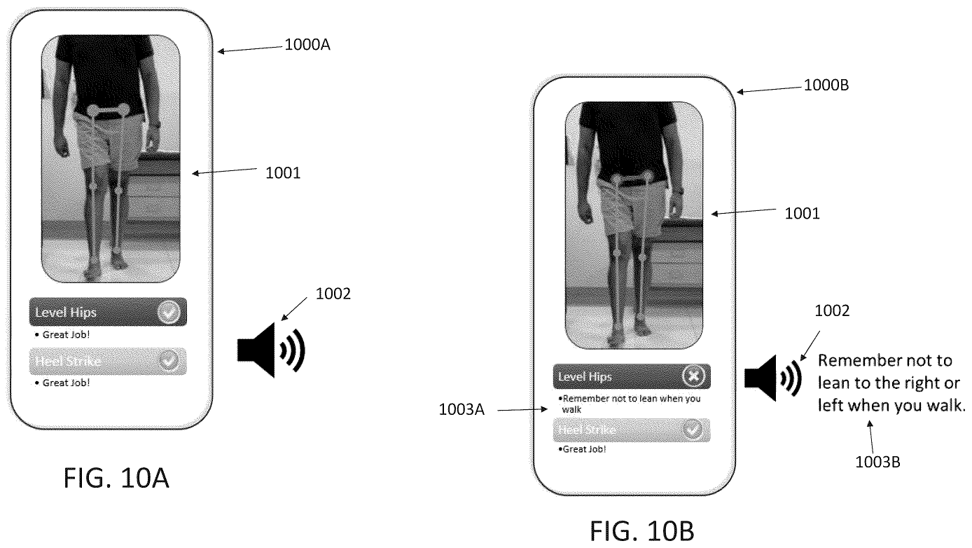

Referring now to FIGS. 10A-10B, an exemplary analysis screen 1000A, 1000B of the system 100 provided according to the present invention is illustrated. The analysis screen 1000A, 1000B may be displayed on a display 1001 of the device through the application after the processing system 120 has constructed the digital wireframe model approximating the patient's skeleton and determined whether one or more gait abnormalities exist. As can be seen in FIG. 10A, which shows the determined anatomical angle being the previously described pelvic inclination, the processing system 120 has determined that the pelvic inclination does not deviate from the defined value by the threshold value and thus does not provide a suggestion to correct any determined gait abnormalities. The processing system 120 also determines that the strike type of the analyzed gait is a heel strike type, which is the healthy strike type. When the processing system 120 determines that no gait abnormalities are observed, the processing system 120 may be configured to output a signal to generate the analysis screen 1000A illustrated in FIG. 10A indicating that the gait is normal and presents affirmations to indicate to the patient that the gait was a healthy gait, i.e., by presenting text and also playing a pre-recorded message that says “Great Job” through a speaker 1002. The system 100 may also be configured to play a pre-recorded message that indicates to the patient that they are walking with the appropriate form, which may be played while images are being captured by the imaging device 111.

FIG. 10B, on the other hand, illustrates an analysis screen 1000B where one or more gait abnormalities is determined to exist. Particularly, as can be seen, the processing system 120 determined the pelvic inclination to deviate from the defined value by more than the threshold value, which indicates that the patient is not walking level. The processing system 120 thus causes the system 100 to provide suggestions 1003A, 1003B to correct the observed gait abnormality based on the anatomical angle (pelvic inclination) that deviates from the defined value by the threshold value. In the case of FIG. 10B, the processing system 120 causes text 1003A to be displayed and/or audio 1003B to be output indicating to the patient that the patient should not lean to the right or left when walking. The provided suggestions 1003A, 1003B thus help the patient identify the abnormality in their gait and how they should be walking to correct the gait abnormality.

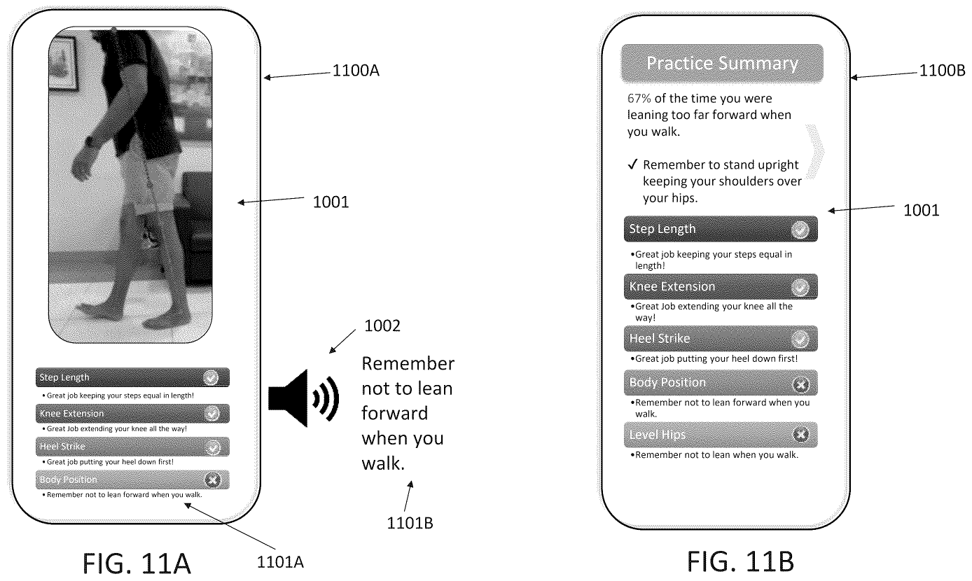

FIGS. 11A and 11B illustrates another exemplary embodiment of analysis screens 1100A, 1100B that the system 100 provided according to the present invention can display. As can be seen in FIG. 11A, the system 100 can determine that the gait abnormality is the patient was leaning forward while walking and provide the suggestions 1101A, 1101B that the patient should not lean forward when walking, in text and/or audio form as shown. FIG. 11B illustrates that the system 100 can also provide an analysis summary screen 1100B that summarizes the analysis of the patient's gait. The analysis summary screen 1100B can show the determined anatomical angle(s) and/or other relevant physiological markers, highlight where gait abnormalities exist, and also show the suggestions to correct the determined gait abnormalities. The analysis summary screen 1100B can thus be a useful way to display suggestions to a patient user on how the patient should focus on altering their gait to address the measured gait abnormalities.

In some embodiments, the system 100 can be configured to present the suggestion as a video demonstrating how the patient should be walking using the patient's own image. For example, the processing system 120 may be configured to determine the one or more gait abnormalities exist and provide the suggestion by generating a demonstration video of the patient walking without the one or more gait abnormalities. The processing system 120 may generate the demonstration video, for example, by using generative artificial intelligence and/or machine learning to generate the demonstration video from the one or more captured images from the image capture system 110. The processing system 120 may be configured to utilize one or more video generation techniques to generate the demonstration video, with one such technique being known as MIMO (Mimic anyone anywhere in complex Motions with Object interactions).

For example, the processing system 120 may be configured to generate the demonstration video from the one or more images used to determine the hip inclination illustrated in FIG. 11A, with the demonstration video being generated to show the patient walking with the hips level, i.e., without the patient leaning forward when walking. In this respect, the processing system 120 can be configured to generate the suggestion as a demonstration video that the patient can watch in order to see what a healthy gait looks like for the patient and try to mimic the healthy gait shown in the demonstration video.

The system 100 can also be configured to store analyses in the memory for future review. In some embodiments, each stored analysis is associated with the particular image(s)/video that the system 100 used to produce the analysis; the associated image(s)/video can also be stored in the memory for future review. Other potentially useful information, such as the date and time of the recording, can also be associated with the analysis and stored in the memory. The system 100 storing analyses in the memory allows a patient and/or healthcare provider to review previously analyzed gaits and determine if the patient's gait has changed over time, which also allows the patient and/or healthcare provider to determine what, if any, further corrective actions may be needed.

Similarly, the system 100 can be provided to utilize an assessment algorithm to determine if any of the anatomical angles have changed over a time interval and determine if the changed anatomical angle(s) has changed in a manner that indicates healing. For example, the system 100 may utilize the assessment algorithm to compare the determined anatomical angle(s) of each stored analysis to the previously saved analysis and determine if the change in the angle(s) is directed closer to or further away from the defined value. If the system 100 determines that the change in the anatomic angle(s) is directed further away from the defined value, which indicates a worsening of the gait abnormality, the system 100 may be configured to issue an alert that the one or more gait abnormalities is not correcting and, in some embodiments, provide a further suggestion indicating how to correct the one or more gait abnormalities. Alternatively, if the system 100 determines that the change in the angle(s) is directed towards the defined value, which indicates correction of the gait abnormality, the system 100 may be configured to issue an alert that the one or more gait abnormalities is correcting, which may encourage the patient and/or healthcare provider to continue the gait correcting actions. The assessment algorithm may be used, for example, by the processing system 120.

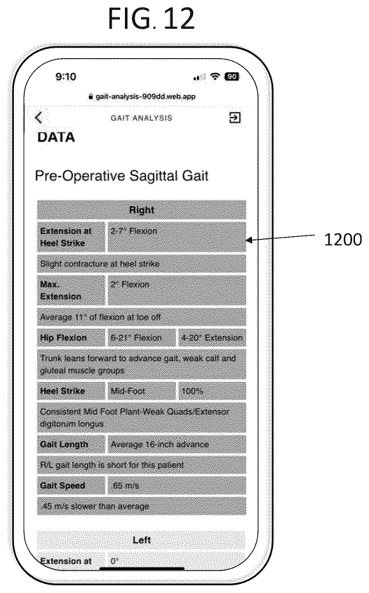

While the previously described examples of FIGS. 2-9 have been with respect to pre-intervention gait analysis, the system 100 provided according to the present invention can also be used for post-operative (and/or post-intervention) gait analysis. Previously described FIGS. 10-11, for example, illustrate post-operative gait analysis. Referring now to FIG. 12, an exemplary post-operative gait analysis screen 1200 provided according to the present invention is illustrated. The post-operative gait analysis screen 1200 illustrated in FIG. 12 shows the determined anatomical angles and other physiological markers that the system 100 determined from the recorded image(s) captured by the imaging device(s) 111. The system 100 provides a summary for each determined gait abnormality based on deviations from the respective defined values, as can be seen in the text that is provided under each determined value. In some embodiments, a surgeon or other healthcare provider can select a particular gait abnormality that is determined and the system 100 can provide a suggestion to correct the gait abnormality in the form of text or a link to a resource detailing common practices to correct the gait abnormality, e.g., surgical and/or physical therapy techniques. It should thus be appreciated that the system 100 provided according to the present invention is useful for both pre-operative and post-operative correction of gait abnormalities.

From the foregoing, it should also be appreciated that the present invention provides a method for identifying and correcting gait abnormalities. The method may be performed by the previously described system 100 and includes capturing one or more images of a patient walking, analyzing the captured one or more images, constructing a digital wireframe model approximating the patient's skeleton based on the captured one or more images, determining one or more anatomical angles in the digital wireframe model approximating the patient's skeleton, determining one or more gait abnormalities exists based on the determined one or more anatomical angles deviating from a defined value by a threshold value, and providing a suggestion to correct the one or more determined gait abnormalities based on the one or more anatomical angles that deviate from the defined value by the threshold value.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A system for addressing gait abnormalities, comprising:
an image capture system comprising at least one image capture device configured to capture one or more images of a patient; and
a processing system comprising at least one processor operatively coupled to the image capture system, the processing system being configured to:
analyze the one or more images of the patient;
construct a digital wireframe model approximating a skeleton of the patent based on the one or more images;
determine one or more anatomical angles in the digital wireframe model;
determine one or more gait abnormalities exists based on the determined one or more anatomical angles deviating from a defined value by a threshold value; and
provide a suggestion to correct the one or more determined gait abnormalities based on the one or more anatomical angles that deviates from the defined value by the threshold value.

2. The system of claim 1, further comprising a display operatively coupled to the processing system, wherein the processing system is configured to provide the suggestion by outputting a visual signal to the display that causes the display to show at least one picture and/or text presenting the suggestion.

3. The system of claim 2, wherein the visual signal causes the display to display a video presenting the suggestion.

4. The system of claim 1, further comprising an audio device operatively coupled to the processing system, wherein the processing system is configured to provide the suggestion by outputting an audio signal to the audio device that causes the audio device to create a sound presenting the suggestion.

5. The system of claim 1, wherein the processing system is configured to analyze a plurality of images of the patient to construct the digital wireframe model.

6. The system of claim 5, wherein the plurality of images are part of a video captured by the at least one image capture device.

7. The system of claim 1, wherein the suggestion is derived from a source that includes a plurality of defined suggestions that are each associated with a degree of gait abnormality.

8. The system of claim 7, wherein the processing system is configured to provide a second suggestion to correct the one or more gait abnormalities when the one or more anatomical angles deviates from the defined value by a second threshold value that is greater than the threshold value.

9. The system of claim 1, wherein the one or more anatomical angles comprises at least one of the following:
a hip knee ankle angle;
a flexion angle;
an ankle sagittal angle; or
a pelvic inclination.

10. The system of claim 1, wherein the processing system is further configured to determine at least one of the following based on the one or more images of the patient:
a gait length of the patient;
a strike type of the patient; or
a hip extension of the patient.

11. A method of addressing gait abnormalities, the method comprising:
capturing one or more images of a patient using an image capture system comprising at least one image capture device;
analyzing the one or more images of the patient;
constructing a digital wireframe model approximating a skeleton of the patent based on the one or more images;
determining one or more anatomical angles in the digital wireframe model;
determining one or more gait abnormalities exists based on the determined one or more anatomical angles deviating from a defined value by a threshold value; and
providing a suggestion to correct the one or more determined gait abnormalities based on the one or more anatomical angles that deviates from the defined value by the threshold value.

12. The method of claim 11, wherein the suggestion is provided on a display as at least one picture and/or text presenting the suggestion.

13. The method of claim 12, wherein the suggestion is provided as a video.

14. The method of claim 11, wherein the suggestion is provided by an audio component as a sound presenting the suggestion.

15. The method of claim 11, wherein analyzing the one or more images comprises analyzing a plurality of images of the patient to construct the digital wireframe model.

16. The method of claim 15, wherein the plurality of images are part of a video captured by the at least one image capture device.

17. The method of claim 11, further comprising deriving the suggestion from a source that includes a plurality of defined suggestions that are each associated with a degree of gait abnormality, the plurality of defined suggestions comprising a second suggestion to correct the one or more gait abnormalities when the one or more anatomical angles deviates from the defined value by a second threshold value that is greater than the threshold value.

18. The method of claim 11, wherein the one or more anatomical angles comprises at least one of the following:
a hip knee ankle angle;
a flexion angle;
an ankle sagittal angle; or
a pelvic inclination.

19. The method of claim 11, further comprising determining at least one of the following based on the one or more images of the patient:
a gait length of the patient;
a strike type of the patient; or
a hip extension of the patient.
